(19) United States
(12) Patent Application Publication
(10) Pub. No.: US 2009/0196885 A1
(43) Pub. Date: Aug. 6, 2009

(54) Cancer Treatment

(75) Inventors: Chih-Jung Yao, Taipei (TW); Shuang-En Chuang, Taipei (TW); Gi-Ming Lai, Taipei (TW); Chin-Feng Chan, Taipei (TW)

Correspondence Address: OCCHIUTI ROHLICEK & TSAO, LLP, 10 FAWCETT STREET, CAMBRIDGE, MA, 02138, US

(73) Assignee: National Health Research Institutes, Zhunan Town (TW)

(21) Appl. No.: 12/421,018

(22) Filed: Apr. 9, 2009

Related U.S. Application Data
(62) Division of application No. 11/582,126, filed on Oct. 17, 2006.
(60) Provisional application No. 60/738,834, filed on Nov. 21, 2005.

(51) Int. Cl. A61K 36/06; A61P 35/00

(52) U.S. Cl. 424/195.15

(57) ABSTRACT

This invention relates to a method of treating cancer by administering to a subject in need thereof an effective amount of a mevalonate pathway inhibitor and an effective amount of an Antrodia camphorata extract.

### RELATED APPLICATIONS

This application is a division of and claims priority to U.S. application Ser. No. 11/582,126, filed on Oct. 17, 2006 and U.S. Provisional Application No. 60/738,834, filed on Nov. 21, 2005, the contents of which are incorporated herein by reference in their entirety.

### BACKGROUND

Cancer, a leading fatal disease, features an abnormal mass of malignant tissue resulting from excessive cell division. Cancer cells proliferate in defiance of normal restraints on cell growth, and invade and colonize territories normally reserved for other cells.

Modes of cancer therapy include chemotherapy, surgery, radiation, and combinations of these treatments. Chemotherapy typically involves use of one or more compounds that inhibit cancer cell growth. While many cancer chemotherapeutic agents have been developed, there remains a need for more effective chemotherapy.

### SUMMARY

This invention is based on a surprising discovery that lovastatin (a mevalonate pathway inhibitor) significantly enhances efficacy of an extract of Antrodia camphorata (also named as “Taiwanofungus camphoratus”) in inhibiting the growth of cancer cells.

Thus, one aspect of this invention relates to a method of treating cancer (e.g., liver cancer, glioma, lung cancer, or pheochromocytoma) with a mevalonate pathway inhibitor and an Antrodia camphorata extract. The mevalonate pathway inhibitor can be a 3-hydroxy-3-methylglutaryl-CoA (HMG-CoA) reductase inhibitor, such as a statin compound (e.g., lovastatin, simvastatin, atorvastatin, fluvastatin, or pravastatin).

Another aspect of this invention relates to a composition containing a mevolante pathway inhibitor, an Antrodia camphorata extract, and a pharmaceutically acceptable carrier for treating cancer, as well as the use of such a composition for the manufacture of a medicament for treating cancer.

Details of several embodiments of the invention are set forth in the description below. Other features, objects, and advantages of the invention will be apparent from the description, and also from the claims.

### DETAILED DESCRIPTION

A mevalonate pathway inhibitor enhances the efficacy of an Antrodia camphorata extract in treating cancer. As a result, a lower dose of the Antrodia camphorata extract is required to obtain desired therapeutic efficacy.

With the scope of this invention is a method of treating cancer by administering to a subject in need thereof an effective amount of an Antrodia camphorata extract and an effective amount of a mevalonate pathway inhibitor. The term “treating” as used herein refers to the administration of a composition including active agents to a subject, who has cancer, a symptom of cancer, or a predisposition toward cancer, with the purpose to cure, heal, alleviate, relieve, alter, remedy, ameliorate, improve, or affect the disease, the symptoms of the disease, or the predisposition toward the disease. “An effective amount” as used herein refers to the amount of each active agent which, upon administration with one or more other active agents, is required to confer therapeutic effect. Effective amounts vary, as recognized by those skilled in the art, depending on route of administration, excipient usage, and the co-usage with other active agents.

The term “cancer” refers to cellular tumor. Cancer cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type, or stage of invasiveness. Examples of cancer include, but are not limited to, carcinoma and sarcoma such as leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, breast cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical or liver cancer, and cancer of unknown primary site.

The term “mevalonate pathway inhibitor” refers to a compound that inhibits any enzyme of the mevalonate pathway (e.g., HMG-CoA reductase), thereby blocking the pathway. It can bind to the enzyme to exert the inhibitory activities or can inhibit the enzyme in an indirect manner in the mevalonate pathway. An example of such an inhibitor is a statin compound, e.g., lovastatin, simvastatin, atorvastatin, fluvastatin, or pravastatin. The mevalonate pathway is well known in the art. See, e.g., Biochemical pathways: An atlas of biochemistry and molecular biology; Ed. Gerhard Michal, Wiley-Spektrum, 1998.

Some mevalonate pathway inhibitors are commercially available. For example, lovastatin and simvastatin are active agents of Zocor® and Mevacor® marketed by Merck & Co. Atorvastatin, fluvastatin, and pravastatin are active agents of Lipitor®, Lescol®, and Pravachol® marketed by Pfizer, Novartis, and Bristol-Myers Squibb, respectively.

The term “Antrodia camphorata extract” refers to substance enriched from Antrodia camphorate (a fungus), which is a commercially available traditional Chinese medicine. To prepare an Antrodia camphorata extract, one can use extracting techniques well known in the art. For example, one can suspend dried and pulverized Antrodia camphorate in a solvent or a mixture of two or more solvents for an adequate length of time. Examples of suitable solvents include, but are not limited to, water, methanol, ethanol, methylene chloride, chloroform, acetone, ether (e.g., diethyl ether), and ester (e.g., ethyl acetate). One can then remove the solid residue, e.g., by filtration, to obtain a solution, which may be used as directly or dried to further provide a solid extract.

To practice the treatment method of this invention, an Antrodia camphorata extract and a mevalonate pathway inhibitor can be applied at the same time (e.g., administration of a composition containing both a mevalonate pathway inhibitor and an Antrodia camphorata extract) or at different times (e.g., administration of a composition containing a mevalonate pathway inhibitor before or after administration of a composition containing an Antrodia camphorata extract). Each of the two active agents, independently, can be administered orally, parenterally, by inhalation spray, or via an implanted reservoir. The term “parenterally” as used herein refers to subcutaneous, intracutaneous, intravenous, intramuscular, intraarticular, intraarterial, intrasynovial, intrasternal, intrathecal, intralesional, and intracranial injection, as well as infusion techniques.

An oral composition can be any orally acceptable dosage form including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A sterile injectable composition (e.g., aqueous or oleaginous suspension) can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A topical composition can be formulated in form of oil, cream, lotion, ointment and the like. Suitable carriers for the composition include vegetable or mineral oils, white petrolatum (white soft paraffin), branched chain fats or oils, animal fats and high molecular weight alcohols (greater than C12). The preferred carriers are those in which the active ingredient is soluble. Emulsifiers, stabilizers, humectants and antioxidants may also be included as well as agents imparting color or fragrance, if desired. Additionally, transdermal penetration enhancers may be employed in these topical formulations. Examples of such enhancers can be found in U.S. Pat. Nos. 3,989,816 and 4,444,762. Creams are preferably formulated from a mixture of mineral oil, self-emulsifying beeswax and water in which mixture the active ingredient, dissolved in a small amount of an oil, such as almond oil, is admixed. An example of such a cream is one which includes about 40 parts water, about 20 parts beeswax, about 40 parts mineral oil and about 1 part almond oil. Ointments may be formulated by mixing a solution of the active ingredient in a vegetable oil, such as almond oil, with warm soft paraffin and allowing the mixture to cool. An example of such an ointment is one which includes about 30% almond and about 70% white soft paraffin by weight.

A carrier in a pharmaceutical composition must be “acceptable” in the sense that it is compatible with active ingredients of the formulation (and preferably, capable of stabilizing it) and not deleterious to the subject to be treated. For example, solubilizing agents, such as cyclodextrins (which form specific, more soluble complexes with one or more of active compounds of the extract), can be utilized as pharmaceutical excipients for delivery of the active ingredients. Examples of other carriers include colloidal silicon dioxide, magnesium stearate, cellulose, sodium lauryl sulfate, and D&C Yellow # 10.

The above-mentioned method may further include applying radiation to the subject to be treated. The radiation used in this method may be ionizing radiation or non-ionizing radiation. Ionizing radiation has sufficient energy to interact with an atom and remove electrons from their orbits, causing the atom to become charged or “ionized.” It includes radiation with gamma ray, X-ray, neutrons, electrons, alpha particles, and beta particles. Non-ionizing radiation is electromagnetic radiation that does not have sufficient energy to remove electrons from their orbits. It includes radiation with ultraviolet rays, visible light, infrared light, microwave, and radio waves. The radiation is applied to the subject after administration of an Antrodia camphorata extract and a mevalonate pathway inhibitor.

Suitable in vitro assays can be used to preliminarily evaluate the efficacy of a combination of an Antrodia camphorata extract and a mevalonate pathway inhibitor in inhibiting growth of cancer cells. The combination can further be examined for its efficacy in treating cancer by in vivo assays. For example, it can be administered to an animal (e.g., a mouse model) having cancer and its therapeutic effect are then accessed. Based on the results, an appropriate dosage range and administration route can also be determined.

Without further elaboration, it is believed that the above description has adequately enabled the present invention. The following specific examples are, therefore, to be construed as merely illustrative, and not limitative of the remainder of the disclosure in any way whatsoever. All of the publications, including patents, cited herein are hereby incorporated by reference in their entirety.

### Example

### Preparation of an Antrodia Camphorate Extract

Antrodia camphorate, provided by Well Shine Biotechnology Development Co. Ltd (Taipei, Taiwan), was air dried and pulverized. 300 g of the pulverized fungus was suspended in 6 L of 95% ethanol. The suspension was stirred at room temperature for 48 hours, and then filtered through 4 A filter paper (Toyo Roshi, Japan) to remove insoluble residue. 500 ml of 95% ethanol was used to wash the residue. The filtrate was collected and concentrated by a rotary evaporator at 40-45° C. to 1/10 of the original volume. The concentrate was evaporated to dryness under nitrogen at room temperature to afford an extract weighing 37.4 g.

### Biological Assay:

An in vitro assay was conducted to evaluate the efficacy of a combination of an Antrodia camphorata extract and a mevalonate pathway inhibitor in inhibiting proliferation of cancer cells.

The Antrodia camphorata extract prepared above was dissolved in 95% ethanol to form a stock solution at the concentration of 100 mg/ml. A stock solution of lovastatin in 100% DMSO (10 mM) was also prepared. Before use, the stock solutions were diluted with Dulbecco's Modified Eagle Medium containing 10% fetal calf serum (complete DMEM) to the desired concentrations.

Hep G2 hepatoma cells were seeded in a 96-well culture plate (2000 cells/well) and cultured overnight in 100 μl of complete DMEM. Each of 50 μl aliquots of complete DMEM containing the Antrodia camphorata extract (40-320 μg/ml) and each of 50 μl aliquots of complete DMEM containing lovastatin (2-16 μM) were then concurrently added to different wells of the culture plate. In addition, 100 μl of complete DMEM was used for controls. After incubation for 6 days, the cell number of each well was determined by the sulforhodamine B (a protein binding dye) assay. Briefly, the cells were fixed in 10% trichloroacetic acid and stained with 0.4% sulforhodamine B. After incubation for 20 min and washing with 1% acetic acid, bound sulforhodamine B was dissolved in 10 mM unbuffered Tris base. The optical density was measured at 562 nm using a microtiter plate reader.

In similar manners, DBTRG-05 MG glioma cells, CL 1-0 lung carcinoma cells, and PC 12 pheochromocytoma cells were also tested against combinations of the Antrodia camphorata extract and the lovastatin.

The results indicate that lovastatin and Antrodia camphorata extract exhibited profound synergistic effect in inhibiting cell growth of Hep G2, DBTRG 05MG, CL 1-0, and PC12. For example, while lovastatin (2 μM) and the Antrodia camphorata extract (20 μg/ml) inhibited growth of Hep G2 cells only by 11.5% and 2.8%, respectively, their combination inhibited the growth by 99.3%. In addition, the cells surviving from the combination treatment showed markedly shrunk morphology.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of treating cancer, comprising administering to a subject in need thereof an effective amount of a mevalonate pathway inhibitor and an effective amount of an Antrodia camphorata extract.

2. The method of claim 1, wherein the cancer is liver cancer.

3. The method of claim 1, wherein the cancer is glioma.

4. The method of claim 1, wherein the cancer is lung cancer.

5. The method of claim 1, wherein the cancer is pheochromocytoma.

6. The method of claim 1, wherein the mevalonate pathway inhibitor is a 3-hydroxy-3-methylglutaryl-CoA reductase inhibitor.

7. The method of claim 6, wherein the 3-hydroxy-3-methylglutaryl-CoA reductase inhibitor is a statin compound.

8. The method of claim 7, wherein the statin compound is lovastatin, simvastatin, atorvastatin, fluvastatin, or pravastatin.

9. The method of claim 8, wherein the cancer is liver cancer.

10. The method of claim 8, wherein the cancer is glioma.

11. The method of claim 8, wherein the cancer is lung cancer.

12. The method of claim 8, wherein the cancer is pheochromocytoma.

13. The method of claim 1, wherein the Antrodia camphorata extract is prepared by extracting Antrodia camphorata plant with ethanol.

14. The method of claim 13, wherein the mevalonate pathway inhibitor is a 3-hydroxy-3-methylglutaryl-CoA reductase inhibitor.

15. The method of claim 14, wherein the 3-hydroxy-3-methylglutaryl-CoA reductase inhibitor is a statin compound.

16. The method of claim 15, wherein the cancer is liver cancer.

17. The method of claim 15, wherein the cancer is glioma.

18. The method of claim 15, wherein the cancer is lung cancer.

19. The method of claim 15, wherein the cancer is pheochromocytoma.
